(19) United States
(12) Patent Application Publication
(10) Pub. No.: US 2008/0227980 A1
(43) Pub. Date: Sep. 18, 2008

(54) Preparation of a 7H-Pyrrolo [2,3-D] Pyrimidine Derivative

(75) Inventors: Robert Portmann, Pratteln (CH); Walter Scherrer, Rheinfelden (CH)

Correspondence Address: NOVARTIS;CORPORATE INTELLECTUAL PROPERTY, ONE HEALTH PLAZA 104/3, EAST HANOVER, NJ, 07936-1080, US

(73) Assignee: NOVARTIS AG

(21) Appl. No.: 11/997,347

(22) PCT Filed: Mar. 8, 2006
(86) PCT No.: PCT/EP06/65052

§ 371 Date: Jan. 30, 2008

Related U.S. Application Data
(60) Provisional application No. 60/706,071, filed on Aug. 5, 2005.

(51) Int. Cl. C07D 487/02

(52) U.S. Cl. 544/280

(57) ABSTRACT

The present invention provides highly efficient methods for the preparation of {6-[4-(4-ethyl-piperazin-1-ylmethyl)-phenyl]-7H-pyrrolo[2,3-d]pyrimidin-4-yl]-((R)-1-phenyl-ethyl)-amine (I).

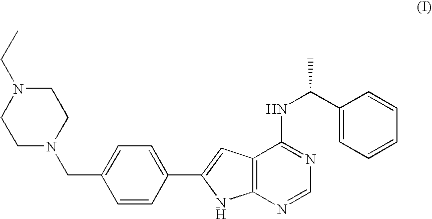

### BACKGROUND OF THE INVENTION

1. Field of the Invention

The present invention provides highly efficient methods for the preparation of {6-[4-(4-ethyl-piperazin-1-ylmethyl)-phenyl]-7H-pyrrolo[2,3-d]pyrimidin-4-yl]-((R)-1-phenyl-ethyl)-amine.

2. Description of Related Art

7H-Pyrrolo[2,3-d]pyrimidine derivatives exhibit a wide array of biological activities. WO 03/013541 describes processes for preparing 7H-pyrrolo[2,3-d]pyrimidine derivatives, including {6-[4-(4-ethyl-piperazin-1-ylmethyl)-phenyl]-7H-pyrrolo[2,3-d]pyrimidin-4-yl]-((R)-1-phenyl-ethyl)-amine. One approach to the synthesis of 7H-pyrrolo[2,3-d]pyrimidine derivatives as described in WO 03/13541 produces an intermediate containing a benzyl chloride moiety, which represents a major safety and hygiene issue. The development of an efficient, safe, and ecologically friendly method for the preparation of 7H-pyrrolo[2,3-d]pyrimidine derivatives by avoiding this type of intermediate still constitutes an important challenge.

### SUMMARY OF THE INVENTION

The present invention provides methods for the efficient preparation of {6-[4-(4-ethyl-piperazin-1-ylmethyl)-phenyl]-7H-pyrrolo[2,3-d]pyrimidin-4-yl]-((R)-1-phenyl-ethyl)-amine, which has the following formula I:

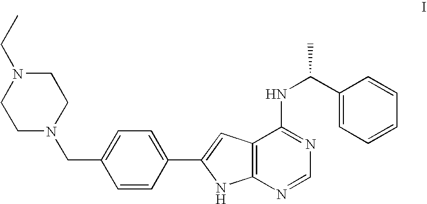

or a salt of the compound, or a solvate or hydrate of the compound, or a salt or a solvent or a hydrate of a salt.

One method of the present invention involves preparing the compound of formula I by the reduction of the amide moiety of (4-ethyl-piperazin-1-yl)-{4-[4-((R)-1-phenyl-ethylamino)-7H-pyrrolo[2,3-d]pyrimidin-6-yl]-phenyl}-methanone, which has the following formula IV:

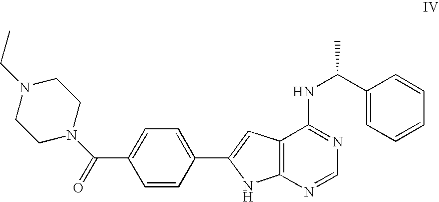

Another method of the present invention involves preparing the compound of formula I by two consecutive reductive amination steps starting from 4-[4-((R)-1-phenyl-ethylamino)-7H-pyrrolo[2,3-d]pyrimidin-6-yl]benzoic acid ethyl ester, which has the following formula II:

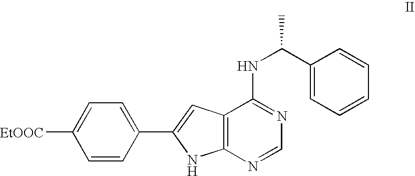

Other objects, features, advantages and aspects of the present invention will become apparent to those skilled in the art from the following description and appended claims. It should be understood, however, that the following description, appended claims, and specific examples, while indicating preferred embodiments of the invention, are given by way of illustration only. Various changes and modifications within the spirit and scope of the disclosed invention will become readily apparent to those skilled in the art from reading the following.

### DETAILED DESCRIPTION OF THE INVENTION

As described above, the present invention provides highly efficient processes for the manufacture of {6-[4-(4-ethyl-piperazin-1-ylmethyl)-phenyl]-7H-pyrrolo[2,3-d]pyrimidin-4-yl]-((R)-1-phenyl-ethyl)-amine.

One method of the present invention comprises reducing the amide of the compound of formula IV. The reduction occurs by using lithium aluminum hydride to produce the compound of formula I. In one embodiment, the compound of formula IV is added to a tetrahydrofuran solution of lithium aluminum hydride. The mixture is subsequently heated to achieve full conversion. After conversion is complete, filter aid and water are added and the solids are removed by filtration. The filter cake is washed with tetrahydrofuran. The filtrate is subsequently concentrated and isopropanol is added. The precipitated product is isolated by filtration.

Another method of the present invention comprises the preparation of the compound of formula I by two consecutive reductive amination steps. In the first step, a solution of lithium aluminum hydride in tetrahydrofuran is added to a solution of 1-ethylpiperazine in tetrahydrofuran. The compound of formula II is subsequently added to the solution. The mixture is subsequently heated to achieve full conversion. After conversion is complete, filter aid and water are added and the solids are removed by filtration. The filter cake is washed with tetrahydrofuran. Acetic acid is added to the filtrate and the solution is concentrated. In the first reductive amination step, yielding the compound of formula I as the main product, the compound 4-[4-((R)-1-phenyl-ethylamino)-7H-pyrrolo[2,3-d]pyrimidin-6-yl]-benzaldehyde, which has the following formula V is formed as a by-product:

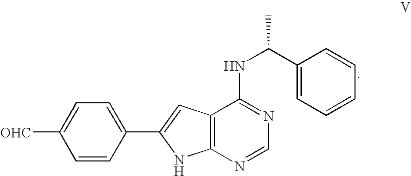

In the second reductive amination step, sodium borohydride is added to the solution thus also transforming compound V, a by-product of the first reductive amination step, to compound I by reaction with the excess of 1-ethylpiperazine remaining from the first reductive amination step. After adding water and removing tetrahydrofuran, the compound of formula I is precipitated by adjusting the pH to about 8 to about 9 and isolated by filtration. The filter cake is washed with water. The moist filter cake is added to a solution of water, ethyl acetate and acetic acid. While the compound of formula I gets dissolved, most of the by-products remain undissolved and are removed by filtration. The filter cake is washed and the filtrate is extracted with ethyl acetate. The pH of the aqueous solution is first adjusted to about 6 to about 6.5 and subsequently readjusted to a pH of about 8 to about 9 by the addition of sodium hydroxide solution. The precipitated compound of formula I is isolated by filtration. The filter cake is washed with water followed by isopropanol. The moist filter cake is added to isopropanol and after stirring, isolated by filtration, washed with water and dried.

The compound of formula IV may be prepared by using the compound of formula II as the starting material. In one embodiment, the compound of formula II is first converted to 4-[4-((R)-1-phenyl-ethylamino)-7H-pyrrolo[2,3-d]pyrimidin-6-yl]benzoic acid, which has the following formula III:

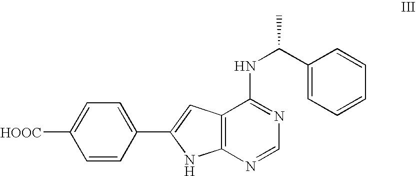

and subsequently reacting the compound of formula III with N,N′-carbonyldiimidazole followed by 1-ethylpiperazine to produce the compound of formula IV.

In one embodiment, the conversion from the compound of formula II to the compound of formula III can be completed using lithium hydroxide monohydrate. The solvents used are ethanol and water. After full conversion, the product is precipitated by adjusting the pH to about 3.3 to about 3.8. The compound of formula III is isolated by filtration.

In one embodiment, the conversion of the compound of formula III to the compound of formula IV is completed by first dissolving the compound of formula III in N,N-dimethylformamide and subsequently adding N,N′-carbonyldiimidazole followed by 1-ethylpiperazine. The reaction mixture is concentrated and after adding an aqueous solution of lithium hydroxide monohydrate in order to keep traces of unreacted compound of formula III dissolved, the product is precipitated by the addition of water and isolated by filtration to yield the compound of formula IV. An optional step includes seeding the reaction solution after addition of part of the total amount of water with the compound of formula IV to produce the compound of formula IV.

One embodiment of the present invention involves preparing the compound of formula I by first converting the compound of formula II

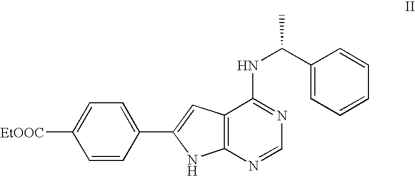

to the compound of formula III

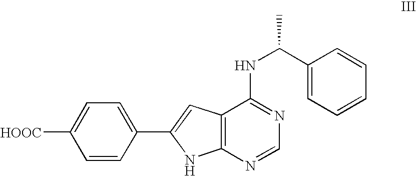

and subsequently reacting the compound of formula III with N,N′-carbonyldiimidazole followed by 1-ethylpiperazine to produce the compound of formula IV

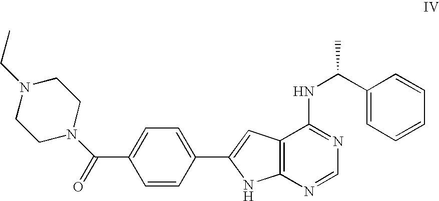

and converting the compound of formula IV to the compound of formula I.

The following examples are intended to illustrate the invention and are not to be construed as being limitations thereof. If not mentioned otherwise, the processes described herein above are conducted under inert atmosphere, preferably under nitrogen atmosphere. All evaporations are performed under reduced pressure, preferably between about 5 and 600 mbar. The structure of products and starting materials is confirmed by standard analytical methods, e.g. melting points (mp) and spectroscopic characteristics (e.g. MS, NMR). Abbreviations used are those conventional in the art.

### EXAMPLE 1

### Preparation of 4-[4-((R)-1-phenyl-ethylamino)-7H-pyrrolo[2,3-d]pyrimidin-6-yl]benzoic acid (Formula III)

4-[4-((R)-1-Phenyl-ethylamino)-7H-pyrrolo[2,3-d]pyrimidin-6-yl]benzoic acid ethyl ester (19.32 g, 50 mmol) (formula II) is suspended in ethanol 94% (97 ml) and stirred, then water is added followed by lithium hydroxide monohydrate (6.29 g, 150 mmol). The suspension is heated to about 55° C. and stirred for about 3 hours. When full conversion is achieved, the mixture is cooled to about 20 to about 25° C. After the addition of xylene (10 ml), the pH is adjusted to about 3.3 to about 3.8 by the addition of sulfuric acid (approx. 84 g, 9% w, 77 mmol). The precipitate is isolated by filtration, washed with water and dried to give the title compound; m.p.>350° C.; MS-ES⁺: (M+H)+=359

### EXAMPLE 2

### Preparation of (4-Ethyl-piperazin-1-yl)-{4-[4-((R)-1-phenyl-ethylamino)-7H-pyrrolo[2,3-d]pyrimidin-6-yl]-phenyl}-methanone (Formula IV)

4-[4-((R)-1-Phenyl-ethylamino)-7H-pyrrolo[2,3-d]pyrimidin-6-yl]benzoic acid (formula III) (7.168 g, 20 mmol) is dissolved in N,N-dimethylformamide (108 ml) at about 40° C. After stirring, the mixture is cooled to about 25 to about 30° C., before the addition of N,N′-carbonyldiimidazole (1.946 g, 12 mmol). The suspension is stirred at about 25 to about 30° C. Another portion of N,N′-carbonyldiimidazole (1.946 g, 12 mmol) is added. Stirring at about 25 to about 30° C. continued for about 1.5 hours. 1-Ethylpiperazine (2.855 g, 25 mmol) is added, then stirring is continued for about 2 hours until full conversion is achieved. The solution is concentrated at a mantle temperature of about 65° C. and at reduced pressure (ca. 25 mbar). After cooling the mixture to about 20 to about 25° C., a solution of lithium hydroxide monohydrate (0.084 g, 2 mmol) in water (5 ml) is added. The mixture is stirred to produce the precipitate.

If no precipitation is observed, the mixture is seeded with 4-ethyl-piperazin-1-yl)-{4-[4-((R)-1-phenyl-ethylamino)-7H-pyrrolo[2,3-d]pyrimidin-6-yl]-phenyl}-methanone (0.01 g) and stirred. Water (67 ml) is added over a period of about 1 hour. The resulting suspension is stirred for another hour. The precipitate is isolated by filtration, washed with water and dried to give the title compound; m.p. 147-155° C. (sintering>112° C.); MS-ES⁺: (M+H)+=455

### EXAMPLE 3

### Preparation of {6-[4-(4-ethyl-piperazin-1-ylmethyl)-phenyl-7H-pyrrolo[2,3-d]pyrimidin-4-yl]-((R)-1-phenyl-ethyl)-amine (Formula I)

A solution of lithium aluminum hydride (14.19 g, 10% w, 37.4 mmol) is added to stirred tetrahydrofuran (85 ml) at about 25° C. (4-Ethyl-piperazin-1-yl)-{4-[4-((R)-1-phenyl-ethylamino)-7H-pyrrolo[2,3-d]pyrimidin-6-yl]-phenyl}-methanone (7.728 g, 17 mmol) is added at about 25° C. over a period of about 1 hour. After stirring, the mixture is heated to about 50° C., then stirred for about 2 hours. When full conversion is achieved, the mixture is cooled to about 25° C. After addition of filter aid (Cellflock 40/Cellulose, 0.935 g), stirring is continued for about 30 minutes. Water (4.1 g, 227 mmol) is added over a period of about 1.5 hours and stirring continues for about another hour. The solids are removed by filtration and the filter cake is washed in portions with tetrahydrofuran. The filtrate is concentrated at a mantle temperature of about 65° C. and about 600-500 mbar. Isopropanol (38.9 ml) is added at normal pressure and the mixture stirred at about 65° C. The filtrate is concentrated by distillation at a mantle temperature of about 65° C. and about 600-350 mbar. Isopropanol (26 ml) is added again at normal pressure and stirred at about 65° C. for about 30 minutes. The suspension is cooled to about 20° C. and stirred for about 1-2 hours. The precipitate is isolated by filtration, washed in portions with isopropanol followed by water and dried to give the title compound; m.p. 243-247° C. (dec.) (sintering>240° C.); MS-ES⁺: (M+H)+=441.

### EXAMPLE 4

### Preparation of {6-[4-(4-ethyl-piperazin-1-ylmethyl)-phenyl]-7H-pyrrolo[2,3-d]pyrimidin-4-yl]-((R)-1-phenyl-ethyl)-amine (Formula I)

A solution of lithium aluminum hydride (15.939 g, 10% w, 42 mmol) is added to a solution of 1-ethylpiperazine (6.85 g, 60 mmol) in tetrahydrofuran (98 ml) at about 25° C. over a period of about 1 hour followed by rinsing with tetrahydrofuran (2 ml). 4-[4-((R)-1-Phenyl-ethylamino)-7H-pyrrolo[2,3-d]pyrimidin-6-yl]benzoic acid ethyl ester (7.729 g, 20 mmol) is added at about 25° C. over a period of about 1 hour, followed by rinsing with tetrahydrofuran (5 ml). The mixture is heated to about 50° C. and stirring is continued for about 1 hour. The mixture is cooled to about 25° C., then filter aid (Cellflock 40/Cellulose, 1.05 g) is added. Water (4.54 g, 252 mmol) is added over a period of about 1.5 hours and stirring continues for about 1 hour. The solids are removed by filtration and the filter cake is washed with tetrahydrofuran. Acetic acid (10.31 g, 171.5 mmol) is added to the stirred filtrate at about 25° C. The solution is concentrated by distilling at a mantle temperature of about 50° C. and about 450-400 mbar. After cooling the concentrate to about 25° C., sodium borohydride (0.227 g, 6 mmol) is added. Stirring is continued and after full conversion, water (54 g) is added. The solution is concentrated by distilling at a mantle temperature of about 50° C. and about 300-150 mbar, until distillation ceases. After cooling to about 25° C., toluene (0.9 ml) is added followed by water (60 ml). Sodium hydroxide solution 30% w (approx. 13 ml, 130 mmol) is added over a period of about 1 hour, until a pH of about 8- about 9 is obtained. Stirring is continued. The precipitated solids are isolated by filtration and washed with water. The moist filter cake (ca. 17 g) is added to a stirred mixture of water (approx. 8 ml), ethyl acetate (5.81 ml) and acetic acid (1.31 g, 21.8 mmol) at about 30° C. Stirring is continued for about 1 h, then water (58 ml) is added and stirring is continued for 3 h. Filter aid (Cellflock 40/Cellulose, 0.1 g) is added. After stirring, the solids are removed by filtration, and the filter cake is washed with water. The filtrate is extracted with ethyl acetate (41.5 ml), the organic layer is separated and the aqueous layer is extracted again with ethyl acetate (41.5 ml). The organic layer is removed and the aqueous layer is filtered. Sodium hydroxide solution 30% w (approx. 0.6 ml, 6 mmol) is added until a pH of about 6- about 6.5 is obtained. After stirring, sodium hydroxide solution 30% w (approx. 1.5 ml, 15 mmol) is added over a period of about 90 minutes, until a pH of about 8- about 9 is reached. Stirring is continued, then the precipitated solids are isolated by filtration, washed in portions with water and isopropanol (15 ml).

The moist solids are added to stirred isopropanol (35 ml) at about 70° C. The suspension is heated to about 80 to about 85° C. and stirred for about 1 hour, then cooled to about 0° C. within about 1 hour and stirred for about another hour. The solids are isolated by filtration, washed in portions with water and dried at about 70- about 80° C. to constant weight to give the title compound; m.p. 243-244° C. (dec.), (sintering>239° C.); MS-ES⁺: (M+H)+=441; HPLC purity>99% area.

### EXAMPLE 5

### Isolation of 4-[4-((R)-1-phenyl-ethylamino)-7H-pyrrolo[2,3-d]pyrimidin-6-yl]-benzaldehyde (Formula V)

A solution of lithium aluminum hydride (7.59 g, 10% w, 20 mmol) is added to a solution of 1-ethylpiperazine (6.85 g, 60 mmol) in tetrahydrofuran (100 ml) at about 25° C. over a period of about 30 minutes. After stirring over night, 4-[4-((R)-1-phenyl-ethylamino)-7H-pyrrolo[2,3-d]pyrimidin-6-yl]benzoic acid ethyl ester (7.729 g, 20 mmol) is added at about 25° C. over a period of about 1 hour, followed by tetrahydrofuran (4 ml) for rinsing. The mixture is heated to about 50° C. over a period of about 1 hour and stirring continues for about 4 hours. The mixture is cooled to about 0° C., then a solution of lithium aluminum hydride (7.59 g, 10% w, 20 mmol) is added. After heating to about 50° C., stirring continues for about 3 hours. After cooling to about 25° C., filter aid (Cellflock 40/Cellulose, 1.10 g) is added. Water (4.75 g, 264 mmol) is added over a period of about 1.5 h and stirring continues for about 2 hours. The solids are removed by filtration and the filter cake is washed in portions with a total of about 60 ml tetrahydrofuran. The filtrate is diluted with water (50 ml), then hydrochloric acid (about 10 ml, 37% w, about 120 mmol) is added until about a pH of 4 is reached. Water (100 ml) is added and the solution is concentrated by distilling at a mantle temperature of about 50° C. and about 300-50 mbar, until about 150 g of residue remains. The resulting suspension is stirred at a mantle temperature of about 50° C. and normal pressure, then the yellow solids are isolated by filtration, washed with water (40 ml) and dried at 60° C. to constant weight to give the title compound. The product is further purified by crystallization from methanol; m.p. 338-339° C. (dec.); MS-ES⁺: (M+H)+=343.

What is claimed is:

1. A method for preparing {6-[4-(4-ethyl-piperazin-1-ylmethyl)-phenyl]-7H-pyrrolo[2,3-d]pyrimidin-4-yl]-((R)-1-phenyl-ethyl)-amine having the following formula I: 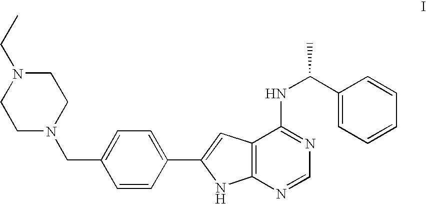
comprising reducing the amide moiety of the compound of formula IV

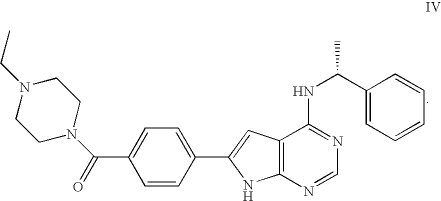

2. The method according to claim 1, wherein lithium aluminum hydride is used to reduce the amide of the compound of formula IV.

3. The method according to claim 1, wherein the compound of formula IV is added to a solution of lithium aluminum hydride in tetrahydrofuran.

4. The method of claim 3 further comprising isolation of the compound of formula IV by filtration.

5. A compound of formula IV:

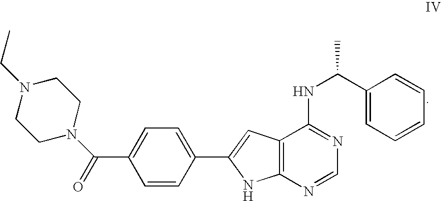

6. A method for preparing {6-[4-(4-ethyl-piperazin-1-ylmethyl)-phenyl]-7H-pyrrolo[2,3-d]pyrimidin-4-yl]-((R)-1-phenyl-ethyl)-amine having the following formula I: 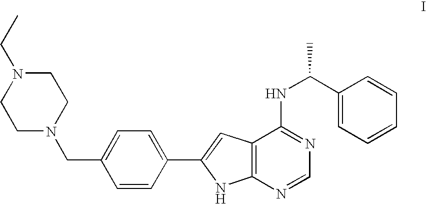
comprising reductive amination of the ester moiety of the compound of formula II

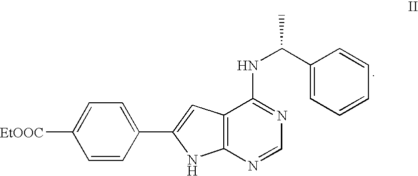

7. The method of claim 6 wherein lithium aluminum hydride and 1-ethylpiperazine are used for the reductive amination of the ester moiety of the compound of formula II.

8. The method of claim 6 wherein the compound of formula II is added to a solution of 1-ethylpiperazine and lithium aluminum hydride in tetrahydrofuran.

9. The method of claim 6 producing as a by-product the compound of formula V:

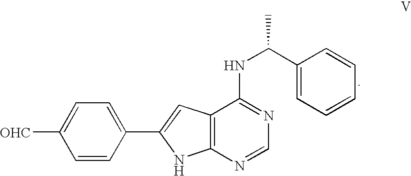

10. A method for preparing {6-[4-(4-ethyl-piperazin-1-ylmethyl)-phenyl]-7H-pyrrolo[2,3-d]pyrimidin-4-yl]-((R)-1-phenyl-ethyl)-amine having the following formula I: 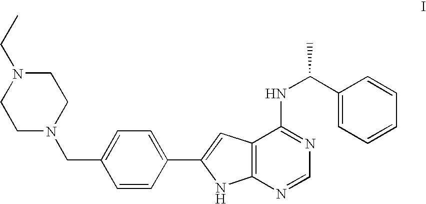
comprising reducing the by-product compound of formula V

11. The method of claim 10 wherein acetic acid and sodium borohydride are used for the reductive amination of the aldehyde group of the compound of formula V.

12. The method of claim 10 further comprising isolation of the compound of formula I by filtration.

13. A compound of formula III:

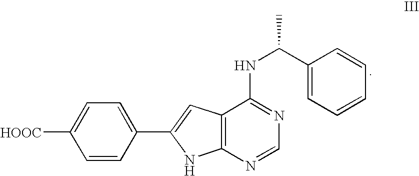

14. A compound of formula V:

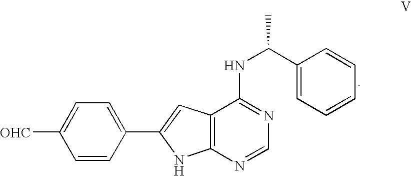

15. A method for preparing the compound of formula IV: 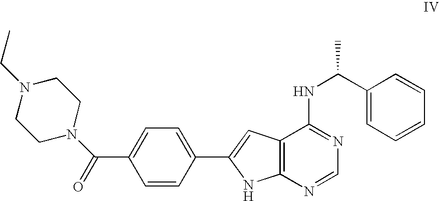
comprising the steps of:
(a) converting the compound of formula II 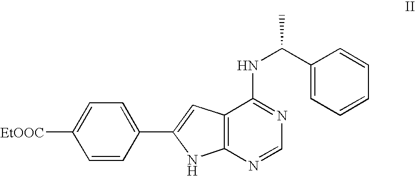
to the compound of formula III 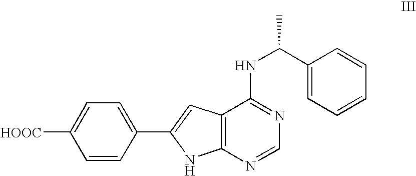
using lithium hydroxide monohydrate in a solvent or a mixture of solvents; and
(b) subsequently reacting the compound of formula III with N,N′-carbonyldiimidazole followed by 1-ethylpiperazine to produce the compound of formula IV.

16. The method of claim 15 further comprising isolation of the compound of formula III by filtration.

17. The method of claim 15 wherein the solvents are ethanol and water.
